(19) United States
(12) Patent Application Publication
(10) Pub. No.: US 2009/0074712 A1
(43) Pub. Date: Mar. 19, 2009

(54) Methods for Treatment and Prevention of Infection

(75) Inventor: Graeme Frith, Reading (GB)

Correspondence Address: ANDRUS, SCEALES, STARKE & SAWALL, LLP, 100 EAST WISCONSIN AVENUE, SUITE 1100, MILWAUKEE, WI, 53202, US

(73) Assignee: Compton, Newbury, Berkshire (GB)

(21) Appl. No.: 11/914,776

(22) PCT Filed: May 19, 2006
(86) PCT No.: PCT/GB06/01849

§ 371 Date: Sep. 30, 2008

Related U.S. Application Data
(60) Provisional application No. 60/689,299, filed on Jun. 10, 2005.

(30) Foreign Application Priority Data
May 19, 2005 (GB) 0510257.9

(51) Int. Cl. A61K 38/20; G01N 33/569; C12Q 1/18; A61K 39/02; A61K 38/21

(52) U.S. Cl. 424/85.2; 435/7.32; 435/32; 424/198.1; 424/85.4; 424/85.7; 424/85.6; 424/85.5

(57) ABSTRACT

Specific cytokines may be used as agents for the treatment and/or prevention of bacterial infection, by binding to components of the bacterial cell membrane and directly killing the bacteria. This finding has a number of applications in screening methods, vaccine production and clearance of bacterial components.

### FIELD OF THE INVENTION

The invention relates to the use of specific innate mammalian cytokines as agents for the treatment and/or prevention of bacterial infection.

### BACKGROUND TO THE INVENTION

Cytokines are small secreted proteins which mediate and regulate immunity, inflammation and hematopoiesis. The largest group of cytokines are those which promote proliferation and differentiation of immune cells. Included within this group are the interleukins, which are cytokines produced by leukocytes, and the interferons, which may be produced by a variety of cell types.

Interferons (IFN) are a family of naturally occurring glycoproteins produced by cells of the immune system of vertebrates, including mammals, birds, reptiles and fish, in response to challenge by agents such as bacteria, viruses, parasites and tumour cells. In humans there are three major classes of interferons. The type I interferons include 14 IFN-alpha subtypes and single IFN-beta, omega, kappa and epsilon isoforms. Type II interferons consist of IFN-gamma and a recently discovered third class consists of IFN-lambda with three different isoforms.

It has been known for some time that interferons are capable of stimulating B-cell and T-cell mediated immune responses.

Interferons alpha and beta were originally described in viral infections and play a prominent role in inhibiting viral infection.

It has been suggested that interferons alpha and gamma may be used in order to treat bacterial infection by promoting an appropriate host cellular immune response in the host organism infected with bacteria. Interferon is used purely for the purposes of stimulating the immune response and actual clearance of the bacterial infection is effected by the subsequent immune system and immune cells of the host.

U.S. Pat. No. 5,817,307 describes treatment of bacterial infection with oral interferon-α. Interferon is administered at a low oral dose in order to potentiate disease-corrective immune responses. The authors state that stimulation of the immune response by oral contact with low dosage interferon is believed to assist the body in fighting bacterial infection. However, there is no indication of the types of bacterial infection which can be treated with IFN-α, nor a potential specific mechanism.

WO 98/33517 also suggests the use of IFN-α subtypes in therapy of bacterial or parasitic infection. Again, IFN-α is stated to be used for the purpose of enhancing immune response, in this case a T cell mediated response. There is no specific disclosure of the types of bacterial and parasitic infections that can be treated with IFN-α. Following treatment with IFN-α to stimulate a T cell mediated response it is postulated that actual clearance of the bacterial or parasitic infection will be mediated by immune cells of the host.

Giosue, S. et al. Am. J. Respir. Crit. Care Med. 158: 1156-1162, 1998 report clinical studies into the effects of aerosolized IFN-α as an immunoadjuvant for pulmonary tuberculosis in humans. Condos, R. et al. (Infect. Immun. 71: 2058-2064, 2003; Lancet 349: 1513-1515, 1997) report the use of aerosolized IFN-γ as an immunoadjuvant in the treatment of pulmonary tuberculosis. In all of these studies IFN is administered for the purposes of stimulating host immune response to bacteria (M. tuberculosis).

The inventor has now demonstrated that certain interferons and other cytokines are capable of binding to native outer membrane vesicles (NOMVs) and specifically bacterial lipopolysaccharides and/or other membrane components and exhibit direct killing of bacteria.

The invention therefore relates to the use of cytokines, and particularly interferons, to mediate direct killing or neutralisation of bacteria.

As discussed above, it is known in the art that interferons are capable of stimulating both B-cell and T-cell mediated immune responses. The inventor has now surprisingly shown that interferons from a variety of different host/cellular sources, and other cytokines, are capable of binding to bacterial NOMVs and specifically bacterial lipopolysaccharides and mediating direct killing of bacterial cells in a dose-responsive manner in the absence of any further components of the immune system, and specifically in the absence of B cells, T cells, phagocytes, serum, complement or any other cells involved in mediating host immune responses.

### SUMMARY OF THE INVENTION

The invention derives from the novel finding that cytokines are capable of binding directly to bacterial lipopolysaccharide and/or other components of the bacterial membrane.

In a first aspect the invention provides a method of treating or preventing bacterial infection in a mammalian host, the method comprising administering to said host an effective amount of at least one cytokine, wherein the cytokine mediates direct killing of the bacteria.

The invention also relates to use of at least one cytokine in the manufacture of a medicament for use in the treatment or prevention of bacterial infection in a mammalian host, wherein the cytokine mediates direct killing of the bacteria.

“Direct killing” is taken to mean that the cytokine is capable of killing the bacteria responsible for the infection in the absence of any further components of the host immune system, such as B cells, T cells or complement. “Direct killing” may be tested in an in vitro killing assay, such as that described in the accompanying examples. “Killing” is taken to mean bacterial cell lysis rather than a bacteristatic effect.

In a second aspect the invention provides a method of neutralising and/or removing from circulation circulating lipopolysaccharides (LPS, also known as endotoxin) or cell membrane-derived vesicles or components comprising lipopolysaccharide in a mammalian subject, the method comprising administering to said subject an effective amount of at least one cytokine. This method may be used to prevent the early stages of LPS-mediated sepsis.

The invention also relates to use of at least one cytokine in the manufacture of a medicament for use in the treatment or prevention of bacterial infection in a mammalian host, wherein the cytokine is capable of neutralising and/or removing from circulation circulating lipopolysaccharides or cell membrane-derived vesicles or components comprising lipopolysaccharide.

In this aspect of the invention “neutralising” of circulating lipopolysaccharides or cell membrane-derived vesicles or components comprising lipopolysaccharide is taken to mean that the lipopolysaccharides or cell membrane-derived vesicles or components comprising lipopolysaccharide are rendered inactive or incapable of triggering adverse physiological effects in the host. The Interferon-Lipopolysaccharide complex may then be removed from general circulation via the kidneys or may be removed with the assistance of an extra-corporeal device or apparatus, for example, as discussed in more detail below.

In preferred embodiments of all aspects of the invention the mammalian host/subject is a human.

In a third aspect the invention provides a vaccine composition comprising pathogenic bacterial cells and/or native outer membrane vesicles (derived from a pathogenic bacteria) and at least one cytokine, wherein the cytokine is capable of binding to a lipopolysaccharide present on the bacterial cell and/or native outer membrane vesicles and/or mediating direct killing of the bacteria (bacterial cells).

The invention also provides a method of preparing a vaccine comprising contacting a preparation comprising live pathogenic bacterial cells and/or native outer membrane vesicles with at least one cytokine, wherein the cytokine is capable of binding to a lipopolysaccharide of the bacterial cells and/or native outer membrane vesicles and/or mediating direct killing of any bacteria (bacterial cells) present in the preparation.

In a fourth aspect the invention provides a method of screening human serum for innate immunity to bacterial infection, the method comprising assaying the serum for the presence of one or more cytokines capable of binding to a lipopolysaccharide present on a bacterial cell or a bacterial cell membrane component and/or mediating direct killing of the bacteria, wherein the presence of one or more said cytokines is taken as an indication of innate immunity to bacterial infection.

In a fifth aspect the invention provides a method of screening human serum for the ability to kill bacterial cells, the method comprising incubating the serum with bacterial cells in the absence of any immune cells or complement then assaying the number of viable bacterial cells present (which will depend upon whether the appropriate cytokines are present, and at appropriate levels, in the serum).

### DETAILED DESCRIPTION OF THE INVENTION

It has long been known that cytokines, such as the interferons and the interleukins, play a central role in regulation of the immune response to infection. Cytokines contribute to adaptive immunity by regulating proliferation and differentiation of immune cells. Certain cytokines also contribute to innate or non-specific immunity. In particular, interferons are known to inhibit viral replication and promote activity of phagocytic cells and also exhibit anti-tumour activity.

The inventor has now surprisingly demonstrated that certain cytokines, including but not limited to the interferons INF alpha, beta and gamma, are capable of directly killing bacterial cells via a cellular and complement-independent mechanism. Thus, specific cytokines themselves provide a rapidly responding and broadly effective innate anti-bacterial response.

A first aspect of the invention therefore relates to the direct use of specific innate mammalian cytokines as agents for the treatment of, and prevention of infection by, bacteria. The invention provides methods of direct treatment of infected subjects/patients with medicaments comprising specific cytokines to rapidly kill blood-bourne, infected tissue dwelling or mucosal-dwelling (e.g. lung and nasopharyngeal tract) bacteria and to neutralise or direct for removal circulating LPS or cell membrane-derived vesicles or components comprising LPS. Such treatment may be particularly useful in overcoming antibiotic resistant bacterial strains and isolates, in the treatment of rapidly disseminating bacterial infection, bacterial biothreat agents or in the treatment of emerging bacterial infectious diseases for which no vaccines are available.

The invention relates to use of cytokines which are capable of binding to bacterial LPS and/or other cell membrane components and directly killing one or more types of bacteria.

In one embodiment the cytokine may be an interferon. Suitable interferons include, but are not limited to, IFN alpha (all subtypes), IFN beta and IFN gamma. In the case of IFN alpha (IFNα) it is possible to use a mixture of subtypes or an IFNα consensus or individual IFNα subtypes may be used singly, or in any combination.

In a further embodiment the cytokine may be an interleukin. Suitable interleukins include, but are not limited to, IL-1a, IL-4, Il-5 and Il-6.

Cytokines, and in particular interferons, may be administered singly or in combination. Combinations of cytokines may be administered sequentially or simultaneously in order to provide a broader spectrum treatment, for example in the absence of specific knowledge of the causative bacterium.

The cytokines may be isolated and purified from natural sources or may be recombinantly synthesised, for example. Suitable “cytokines” also include non-natural, synthetic or altered forms such as, inter alia, mutants, peptides, chimeras, truncated forms or fusion proteins in which biological function is conserved and also cytokine mimetics which exhibit substantially similar biological function. “Biological function” in this context is defined as the ability to bind to bacterial LPS or other cell membrane components and/or to directly kill bacterial cells. The cytokines may be modified for pharmacological reasons. For example, to increase half life the cytokines may be pegylated. Hybrid forms of cytokines, in which parts of individual cytokines have been linked together, are also considered to be encompassed by the term “cytokine”, provided they exhibit substantially similar biological function (as defined above). For example, in U.S. Pat. No. 6,403,077, chimeric proteins consisting of a cytokine bonded to an inactive polypeptide are described, which increases the circulating half life of the cytokine. This reference is incorporated herein in its entirety.

The invention also encompasses the use of molecules which mimic the binding of cytokines to bacterial LPS or other cell membrane components and also exhibit the effects of directly killing bacterial cells and/or neutralising or directing for removal circulating LPS or cell membrane derived vesicles or components comprising LPS. Such cytokine mimetics may include antibodies or fragments thereof such as F(ab′)₂ fragments, scAbs, Fv and scFv fragments etc., provided that they are capable of mimicking cytokine binding.

The cytokine may be derived from any species, provided that it exhibits the ability to bind to bacterial cellular source LPS or other cell membrane components. In particular, the cytokine may be derived from any mammalian species including, inter alia, human, rat, mouse, sheep, cow etc. The invention encompasses, but is not limited to, use of purified native or recombinant cytokines from non-human mammalian species or humans (as well as peptides, mutants, truncated forms, fusion proteins, chimeras, mimetics etc, derived therefrom, as described in further detail above) in the treatment of bacterial infection in human patients.

The inventor has demonstrated by way of in vitro binding experiments that a broad range of cytokines are capable of binding directly to lipopolysaccharide (LPS) prepared from clinical isolates and vaccine strains of pathogenic bacteria, in pure form or in the form of native outer membrane vesicles (NOMVs). Certain cytokines, particularly IFN αA, are capable of binding to NOMVs much better than to free LPS. This indicates that certain cytokines may be capable of binding to cell membrane components in addition to, or in preference to, LPS. Therefore, according to the invention a given cytokine may be used to treat infection with any bacterium which produces LPS to which the cytokine is capable of binding and/or with any bacterium producing another membrane component to which the cytokine is capable of binding.

In one embodiment the invention relates to use of cytokines to treat or prevent infection with Gram-negative bacteria.

In particular non-limiting embodiments the Gram-negative bacteria may be of the genus: Neisseria, Burkholderia, Yersinia, Franscisella, Escherichia, Salmonella, Shigella, Pseudomonas, Brucella, Legionella, Klebsiella, Vibrio, or Haemophilus.

In one embodiment the bacteria may be Neisseria meningitides, in particular Neisseria meningitidis type B, for which there is a need for an effective vaccine and therapy.

In a further embodiment the bacteria may be Yersinia pestis, the causative agent of bubonic plague.

In a still further embodiment the bacteria may be Burkholderia pseudomallei, the causative agent of melioidosis (Sprague and Neubauer, J. Vet. Med., Vol. 51, pp 305-320, 2004).

In a still further embodiment the bacteria may be Franscisella tularensis, the causative agent of tularaemia.

In a still further embodiment, the bacteria may be Pseudomonas aeroginosa.

The inventor has observed that cytokines exhibit a degree of specificity of binding to LPS from different bacterial species and are therefore expected to mediate killing of different sub-groups of target bacteria.

Binding of cytokines to bacterial LPS can be readily assessed using any suitable technique for measurement of (direct) in vitro binding, such as surface plasmon resonance which can be measured using the (commercially available) BIAcore™ apparatus (see the experimental section below for further details).

Thus, for any given bacterial species it is possible to screen samples of LPS, NOMVs or even whole bacterial cells against a panel of cytokines in order to determine which cytokines are capable of binding at high affinity, in a stable and specific manner. Cytokines which bind may then be tested in an in vitro killing assay, such as those described in the accompanying examples, in order to confirm that the cytokine mediates bacterial killing. In this way it is possible to select the most appropriate cytokine, or combination of cytokines, for formulation into a medicament to treat/prevent any given bacterial infection.

Accordingly, in a further aspect the invention provides a method of screening a test agent for the ability to kill directly a bacterium comprising determining whether the test agent has the ability to bind to LPS from the (cell membrane of the) bacterium.

Preferably, the LPS is found as a constituent of NOMVs or whole bacterial cells.

In one preferred embodiment, surface plasmon resonance is utilised in order to determine whether the test agent binds to LPS. However, any other suitable technique may be employed to measure binding.

In a further aspect, the invention provides a method of screening a test agent for the ability to kill directly a bacterium comprising carrying out a suitable killing assay.

Preferably, the killing assay employed is the in vitro killing assay described in detail in the experimental section. Thus, in one embodiment, the method comprises culturing the bacterial cells in the presence of the test agent and then determining the number of viable cells. The culture may be carried out on any suitable solid media for example. Culture is preferably carried out in the absence of any further components of the immune system, and specifically in the absence of B cells, T cells, phagocytes, serum, complement or any other cells involved in mediating host immune responses. This ensures that only direct killing of the bacterial cells is measured.

Suitable controls may be employed. For example the viable cell count may be compared with that for a sample in which the cells were untreated, or were treated with a reagent, compound, molecule or otherwise which is known not to have a direct killing ability. One example of such a reagent is PBS.

The test agent may be any suitable test agent hypothesised to be capable of mediating direct killing of bacteria. Thus, the methods are used to screen agents which are not previously known to have the ability to mediate direct killing of bacteria. Cytokines and derivatives and mimetics thereof, as discussed in more detail above, represent preferred test agents. The methods of these aspects of the invention allow new agents to be designed which are capable of killing bacteria directly and which therefore may have a number of useful applications (as discussed in greater detail herein).

The bacterium may be any suitable bacterium of interest, for which direct killing may be advantageous. Examples are provided throughout the description and these specific bacteria represent preferred target bacteria according to the methods of the invention.

In a particularly preferred embodiment, the two distinct methods are combined in order to discover potential new therapeutics. Thus, if binding to LPS is found for the test agent in question, the direct killing ability of the test agent can then be confirmed in a suitable killing assay.

Suitable combinations of cytokines and bacteria for the purposes of the present invention include, but are not limited to, IFN alpha (preferably mouse or human), IFN beta (preferably rat or human) or IFN gamma (preferably human) for the treatment/prevention of infection with N. meningitides, preferably N. meningitidis type B. Specific preferred IFNs are described in the experimental section below.

In a further embodiment, mouse IFN alpha consensus or human IFN gamma may be utilised for the treatment/prevention of infection with Pseudomonas aeroginosa, preferably the G38 strain of Pseudomonas aeroginosa.

Cytokines may be formulated into pharmaceutical compositions (medicaments) for use in accordance with the invention, together with suitable pharmaceutically acceptable carriers, diluents or excipients. Suitable pharmaceutically acceptable carriers include, for example, non-toxic salts, sterile water or the like. The carrier may contain other pharmaceutically acceptable excipients for modifying other conditions such as pH, osmolarity, viscosity, sterility, lipophilicity, somobility etc. The formulation should not adversely affect the bioactivity of the cytokine, this being defined as ability to bind to bacterial LPS or other cell membrane components and/or to directly kill bacterial cells.

The terms “treating” or “treatment” as used herein refer to the management or care of a patient for the purposes of combating the bacterial infection, and any disease condition or disorder associated therewith and includes the administration of a medicament according to the invention to prevent the onset of the symptoms or complications (including prophylactic treatment).

Direct prophylactic treatment with specific cytokines can provide rapid protection pre-, post- and during exposure to infection, to compensate for an effective lack of a neutralising antibody response or inability to naturally secrete levels of specific cytokines. Such treatment may be especially suited to infants, elderly and immuno-compromised individuals, failed vaccine responders or individuals exposed to or at high risk of exposure to weaponised pathogens (such as Yersinia pestis for example).

The phrase “effective amount” is taken to mean a therapeutically effective amount. The exact dosage and frequency of administration of a therapeutically effective amount of a medicament according to the invention may depend on such factors as the form of the active substance, the dosage form in which it is administered and route of administration, the particular condition to be treated, the severity of the condition being treated and the age, weight and general physical condition of the patient, as would be appreciated by those skilled in the art.

Suitable dosage forms for direct administration of cytokines include solid dosage forms, for example, tablets, capsules, powders, dispersible granules, cachets and suppositories, including sustained release and delayed release formulations. Liquid dosage forms include solutions, suspensions and emulsions. Liquid form preparations may be administered by intravenous, intracerebral, intraperitoneal, parenteral or intramuscular injection or infusion. Sterile injectable formulations may comprise a sterile solution or suspension of the cytokine in a non-toxic, pharmaceutically acceptable diluent or solvent. Suitable diluents and solvents include sterile water, Ringer's solution and isotonic sodium chloride solution, etc. Liquid dosage forms also include solutions or sprays for intranasal administration.

Aerosol preparations suitable for inhalation may include solutions and solids in powder form, which may be combined with a pharmaceutically acceptable carrier, such as an inert compressed gas.

Also encompassed are dosage forms for transdermal administration, including creams, lotions, aerosols and/or emulsions. These dosage forms may be included in transdermal patches of the matrix or reservoir type, which are generally known in the art.

Pharmaceutical preparations may be conveniently prepared in unit dosage form, according to standard procedures of pharmaceutical formulation. The quantity of active compound per unit dose may be varied according to the nature of the active compound and the intended dosage regime. Generally this will be within the range 0.1 mg to 1000 mg.

For treatment/prevention of infection with N. meningitides and other bacteria which colonise mucosal surfaces, e.g. those of the airways, the medicaments according to the invention may be administered directly to the mucosal surface, for example via aerosol administration. Aerosolized formulations of interferons are known in the art.

As well as direct treatment with exogenously administered cytokines, the invention also relates to modulation of specific host innate cytokine levels to generate a rapidly responding and broadly sterilising antibacterial response by naturally inducing an elevated cytokine status.

For example, a molecule based upon LPS (endotoxin) may be used to stimulate a natural elevated cytokine status. Such a response may be induced rapidly by the LPS or derivative thereof and may be localised or systemic. The molecule may be full length LPS or may comprise the portion of LPS to which the cytokine binds for example. Alternative means of stimulating an elevated cytokine status include, by way of example but not limitation, use of an inactivated virus or a component thereof.

In addition to prevention/treatment of bacterial infection via direct bacterial killing, the invention also relates to use of cytokines to neutralise or direct for removal circulating lipopolysaccharide (LPS).

In one embodiment neutralisation/removal of LPS can take place within the body, following administration of exogenous cytokines or up-regulation of the levels of endogenous cytokines. By way of example, interferons may be used in the treatment of early sepsis: LPS (endotoxin) is removed from circulation by binding to IFN, then rapid clearance of IFN (half life is about 20 minutes in a natural form) from the systemic circulation via the kidney and bladder excretion follows. Exogenous IFNs may be administered to a human (or mammal in the case of veterinary treatment) subject in order to boost the LPS (endotoxin) removal process.

In a further embodiment cytokines (e.g. interferons) may be administered externally to the body to neutralise/remove LPS from blood ex vivo, for example using an extra-corporeal device or apparatus. In this embodiment blood may be circulated within a device or apparatus containing one or more cytokines. LPS present in the blood may thus be neutralised/removed via binding to cytokines within the device/apparatus. The amount of LPS in the blood returned to the systemic circulation will thus be reduced. Suitable apparatus may include an external blood filter containing one or more cytokines.

Neutralisation/removal of LPS may take place in the presence or absence of active bacterial colonization. With certain bacterial infections circulating LPS may persist after bacterial replication/colonisation is under control, or LPS may be released as bacterial cells are killed. This circulating LPS would be hazardous to health unless rapidly and effectively removed.

Therefore, in a second aspect of the invention the ability of cytokines to bind to LPS with high affinity can be employed to neutralise or direct for removal LPS remaining in the circulation after a bacterial infection has been neutralised/brought under control. Neutralisation/removal of LPS occurs as a result of specific binding of one or more cytokines to the LPS. Medicaments comprising cytokines capable of binding to LPS thus represent a new class of “LPS scavengers”.

A third aspect of the invention relates to the use of cytokines in the production of anti-bacterial vaccines based on whole cells or membrane components, such as NOMVs.

Whole cell or whole organism vaccines are generally based either on live attenuated strains which are capable of replication within the host, providing a powerful stimulus to the immune system, without causing a significant illness in immune-competent individuals, or on inactivated or killed whole cells which can no longer replicate within the host.

A major drawback with existing methods of vaccine inactivation, such as treatment with heat, formaldehyde or phenol, is that such treatments can interfere with the conformation of epitopes on accessible antigens. Thus, the epitopes presented following such treatments are generally not accessible when the vaccine is “live”. This leads to ineffective immune responses by vaccinated subjects, since the antibodies raised are raised against epitopes typically not readily available, accessible or naturally present in, or on, the live pathogen.

The ability of cytokines to bind to bacterial LPS and directly kill bacterial cells can be exploited in the production of inactivated whole cell or NOMV-based vaccines. Preparations of whole pathogenic bacterial cells and/or NOMVs are contacted with one or more cytokines capable of binding to LPS present on any bacterial cells in the preparation, resulting in the production of a vaccine composition which effectively represents a whole and “native” bacterial preparation whilst the cytokine performs a second role as an adjuvant. Thus, the immune response is improved because the inactivated whole cell or NOMV-based vaccine is based upon the presentation of epitopes present in the live bacterial cell.

This methodology offers a significant increase in native epitopes accessible to the host immune response, a manufacturing process that can be rapidly optimised and provides favourable economic advantages over other vaccine production strategies.

In this context “pathogenic” bacterial cells refers to a naturally occurring disease-causing strain as opposed to a laboratory-derived attenuated strain.

Any of the cytokines, bacterial cells or combinations thereof identified in connection with the first aspect of the invention may also be used in this aspect of the invention.

In a particularly preferred embodiment the method according to this aspect of the invention can be used in the production of NOMV-based vaccines against Neisseria meningitides, particularly Neisseria meningitidis type B. Production of NOMVs from strains of N. meningitidis is well known in the art (Bjune G. E. et al, 1991. Lancet. 338:1093-1096.) Preparations of NOMVs may contain an amount of whole bacterial cells which must be inactivated prior to administration of the vaccine composition to a host subject. Prior art methods of inactivation rely on treatment with heat, formaldehyde or phenol to inactivate any whole, live bacterial cells present in the preparation of NOMVs.

In a further embodiment, the method according to this aspect of the invention can be used in the production of NOMV-based vaccines against Pseudomonas aeroginosa.

According to the invention, inactivation of any bacterial cells in the NOMV preparation can be achieved by treatment with a suitable cytokine which is capable of binding to LPS or other cell membrane component present on any whole bacterial cells present in the NOMV preparation and mediating killing of the cells. Inactivation via this mechanism has the advantage that it does not destroy native epitopes present on the NOMVS.

Suitable cytokines for inactivation of NOMV-based vaccines against Neisseria meningitides, particularly Neisseria meningitidis type B include IFN alpha (preferably mouse or human), IFN beta (preferably rat or human) or IFN gamma (preferably human).

Suitable cytokines for inactivation of NOMV-based vaccines against Pseudomonas aeroginosa, preferably the G38 strain of Pseudomonas aeroginosa, include mouse IFN alpha consensus or human IFN gamma.

Vaccine compositions produced according to this aspect of the invention can be formulated with well known pharmaceutically acceptable excipients such as glycerol, and phosphate buffered saline, to make vaccine compositions which can be administered to a human or non-human animal subject to elicit an immune response, e. g. by intranasal, subcutaneous or intramuscular administration. Immunisation can be carried out either with single doses, or with multiple doses.

### Screening Assays

The invention further provides screening assays for profiling specific patient or vaccinated person cytokine responses during clinical and vaccine trials, to follow clinical treatments and outcomes of infected patients.

Therefore, in a fourth aspect the invention relates to a method of screening human serum for innate immunity to bacterial infection, the method comprising assaying the serum for the presence of one or more cytokines capable of binding to a lipopolysaccharide present on a bacterial cell or a bacterial cell membrane component and/or mediating direct killing of the bacteria, wherein the presence of one or more said cytokines is taken as an indication of innate immunity to bacterial infection.

Screening for the presence of specific cytokines can be carried out by any suitable assay methodology, such as for example, ELISA, radioimmunoassay etc. The method can be used to determine a cytokine profile for any given individual, which in turn provides an indication of the individuals innate immunity to bacterial infection. The method can be used, for example, to determine patient susceptibility to bacterial infection, before, during or after exposure to a pathogen.

The relative levels of specific cytokines may be determined, since the ability to produce a certain minimum quantity of specified cytokines may contribute to the individual's innate immunity to bacterial infection.

In a fifth aspect the invention provides a method of screening human serum for the ability to kill bacterial cells, the method comprising incubating the serum with bacterial cells in the absence of any immune cells or complement then assaying the number of viable bacterial cells present.

The method according to this aspect of the invention can be used as a bioassay for the innate “killing” ability of patient serum samples, providing an indication of the ability of molecules present in the serum (cytokines) to mediate bacterial killing in the absence of any immune cells or complement. Thus, the number of viable bacterial cells remaining is an indication of the cytokine profile of the serum sample, wherein if cytokines are present the number of bacterial cells can be expected to be significantly reduced or eliminated entirely compared to a control serum with no cytokines present.

The bacterial cells used in the assay can be any suitable bacterial strain. Serum from one individual may be tested against a panel of different bacteria in order to assess the spectrum of innate immunity/killing ability for that individual.

The assay according to this aspect of the invention may be based, for example, on the in vitro serum bactericidal assay described in the accompanying examples.

The invention will be further understood with reference to the following non-limiting experimental examples and figures in which:

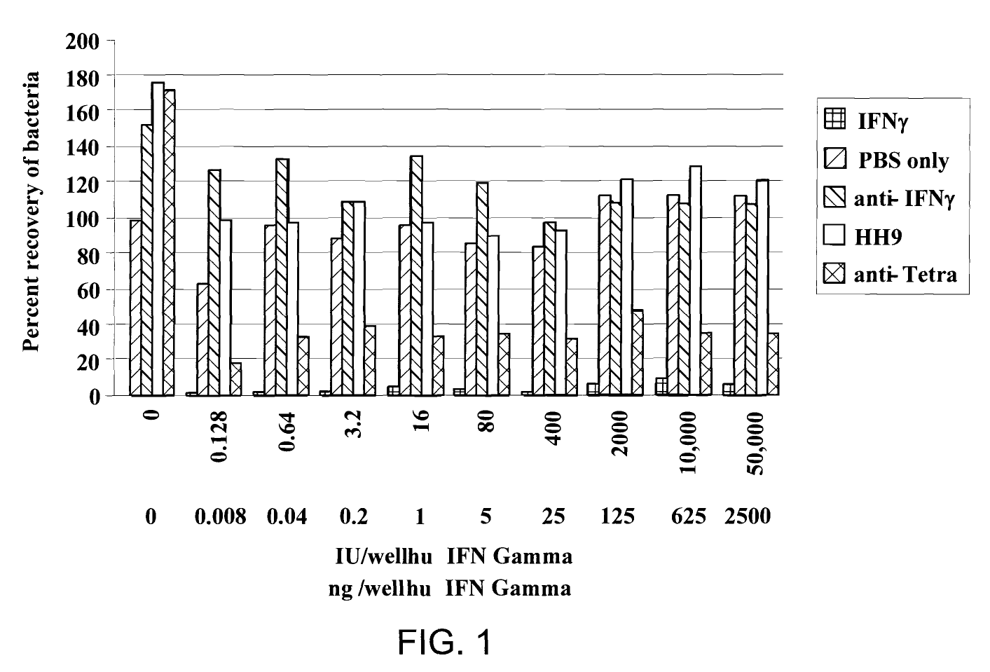

FIG. 1 shows the results from an in vitro bactericidal assay incubation of the Neisseria meningitidis type B clinical isolate 240 101 in the presence of human IFN γ (gamma) and various blocking murine antibodies.

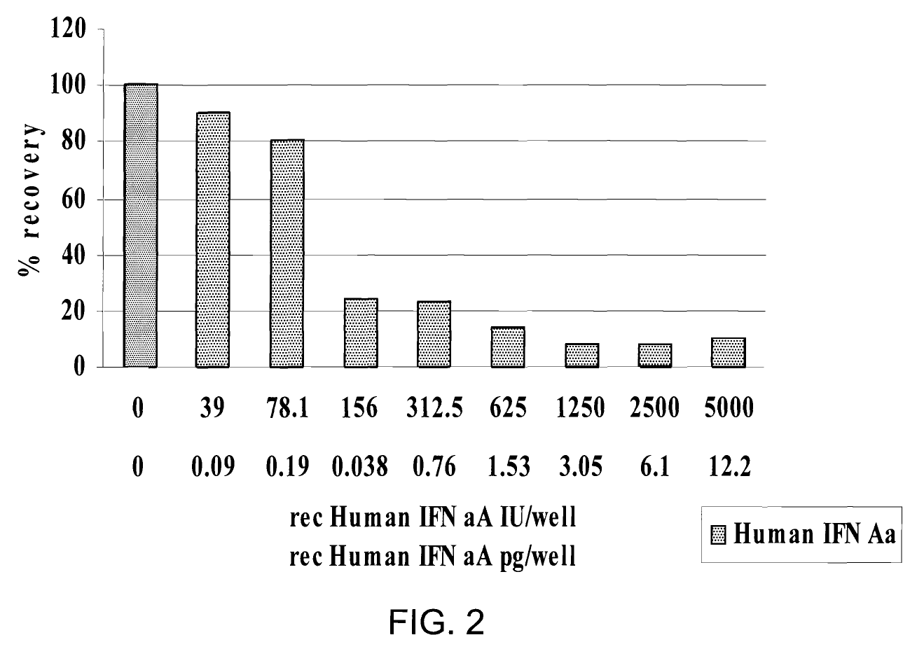

FIG. 2 shows results from an in vitro bactericidal assay incubation of the Neisseria meningitidis type B clinical isolate 240 101 in the presence of human IFN aA (n2).

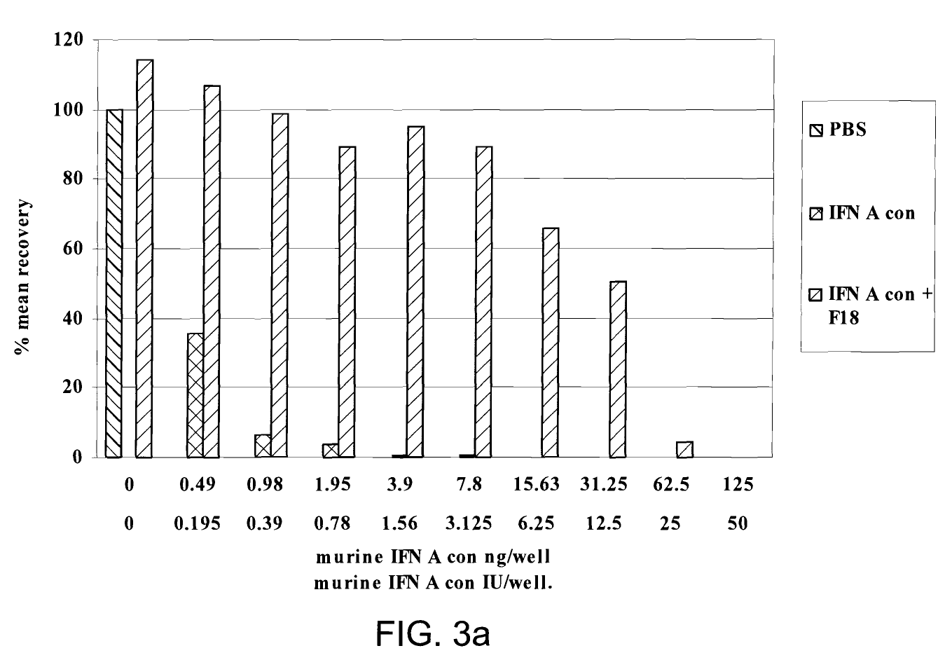

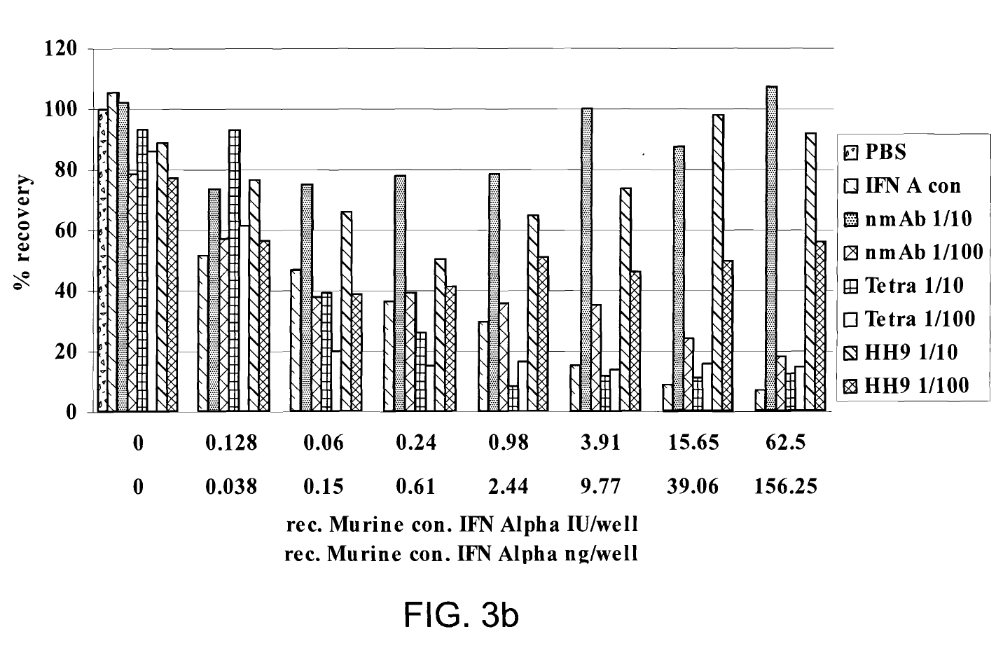

FIG. 3a shows results from an in vitro bactericidal assay incubation of the Neisseria meningitidis type B clinical isolate 240 101 in the presence of murine consensus IFN α in the presence or absence of the murine IFN α neutralising F18 antibody (nmAb).

FIG. 3b shows results from an in vitro bactericidal assay incubation of the Neisseria meningitidis type B clinical isolate 240 101 in the presence of murine consensus IFN α in the presence of various purified murine blocking monoclonal antibodies (mAb) used at 1/10 and 1/100 dilutions.

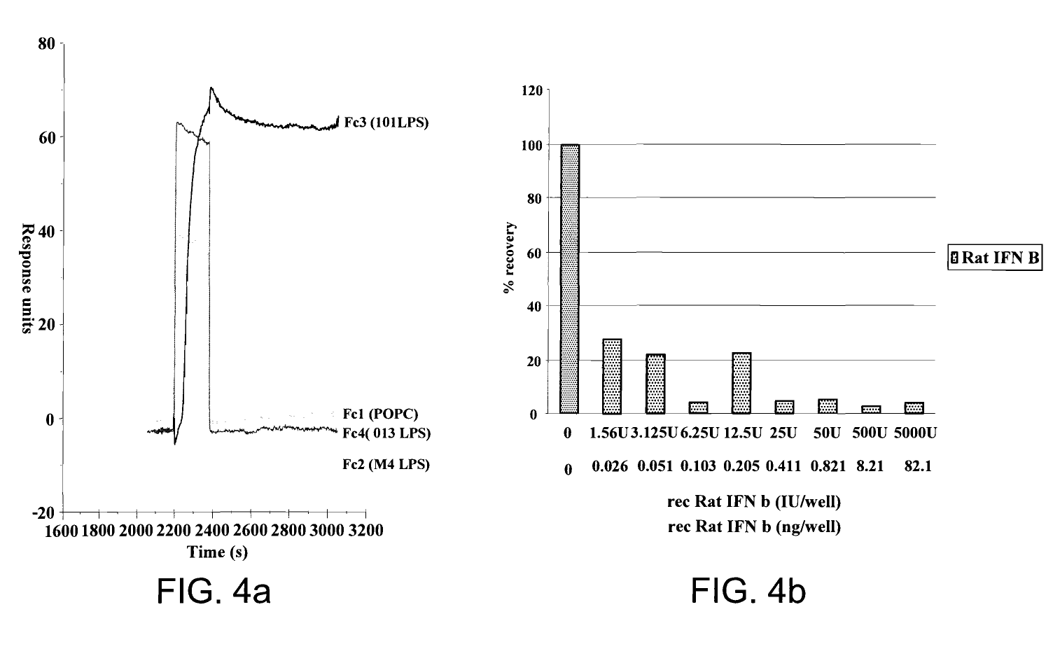

FIG. 4a shows results from the surface plasmon resonance (SPR) BIAcore experiments demonstrating IFN β (beta)(100 nM) specificity of binding to Neisseria meningitidis type B clinical isolate 240 101 LPS (Fc3), as compared to various other purified LPS.

FIG. 4b shows the result from the in vitro bactericidal assay incubation of the 240 101 isolate in the presence of (recombinant) Rat IFN β (beta).

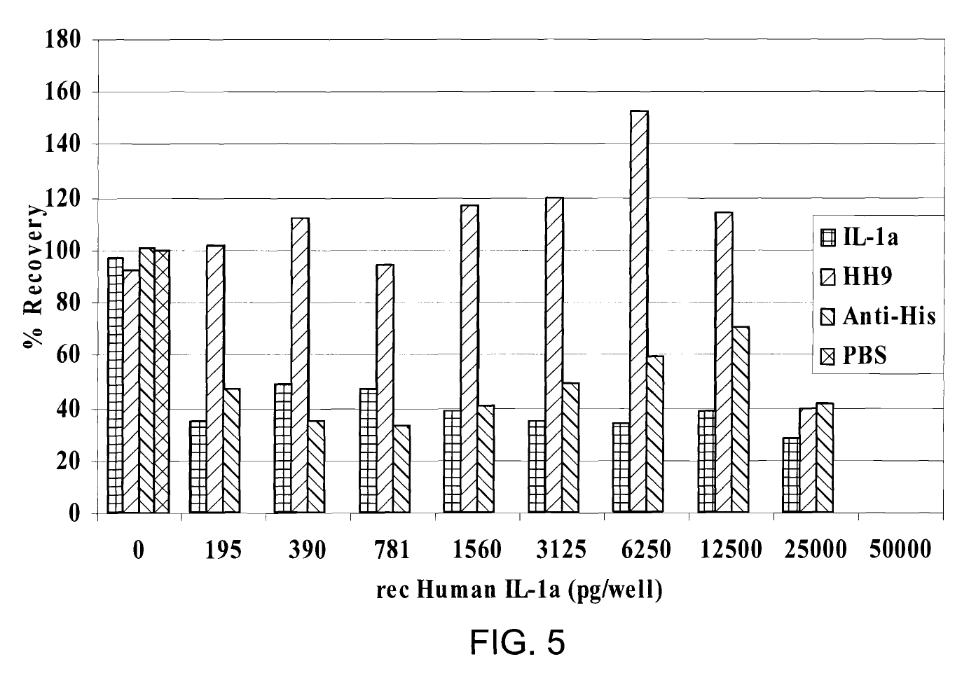

FIG. 5 shows the result from the in vitro bactericidal assay incubation of the Neisseria meningitidis type B mutant 4 incubated in the presence of purified recombinant murine IL-1a and various blocking antibodies.

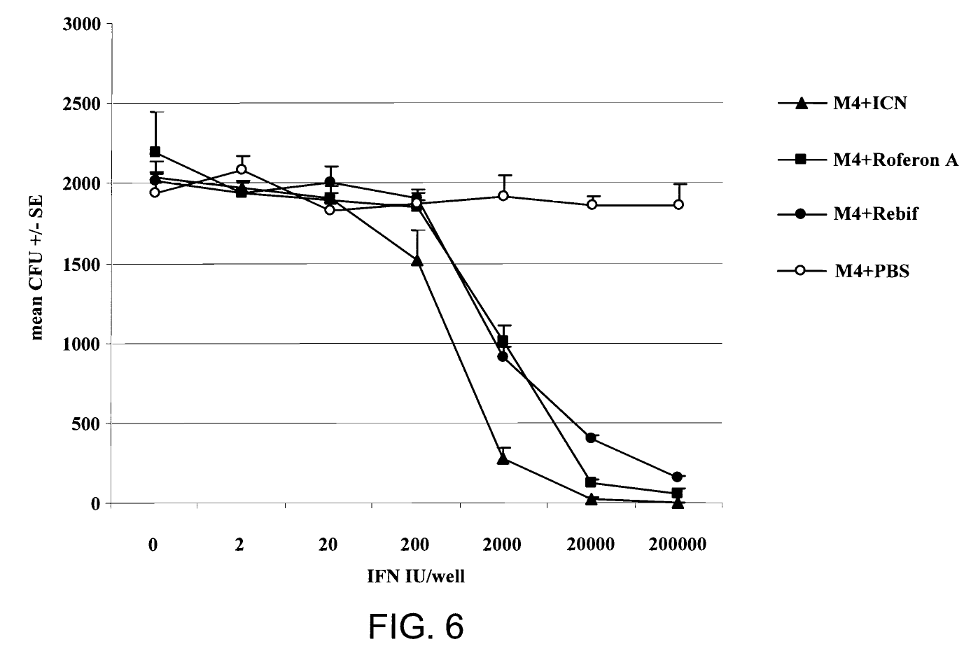

FIG. 6 shows the result from the in vitro bactericidal assay incubation of the short chain LPS Neisseria meningitidis type B 44/76 mutant 4 (M4) with murine consensus IFN a (ICN), human IFN a-2a (Roferon A from Roche) and human IFN b-1a (Rebif from Serono). A PBS control is included.

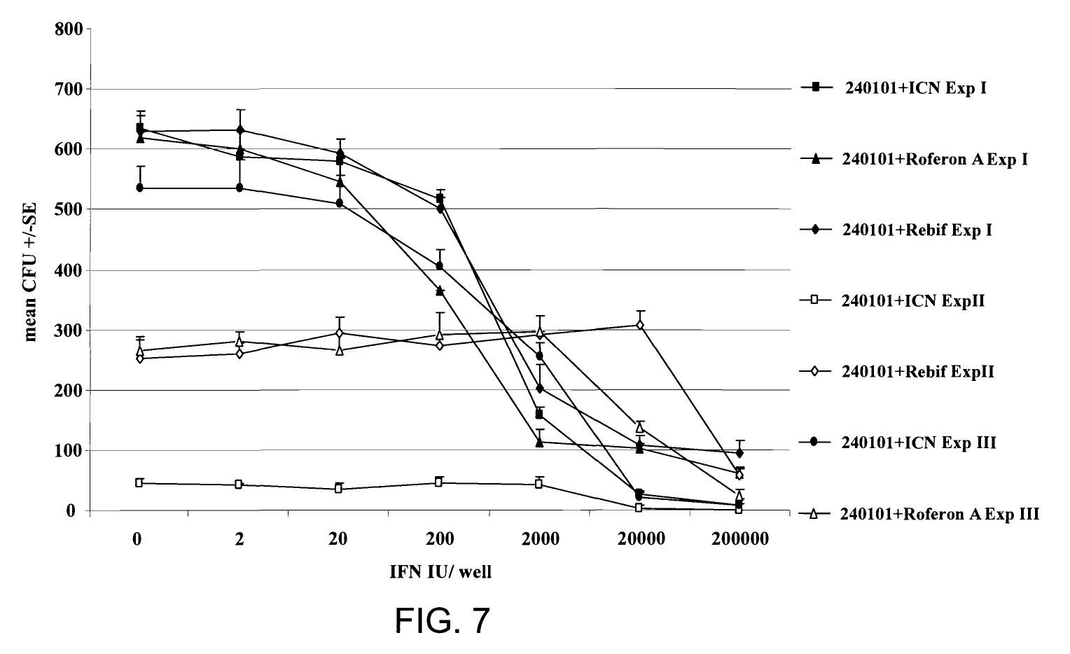

FIG. 7 shows the result from the in vitro bactericidal assay incubation of the Neisseria meningitidis type B clinical isolate 240 101 with murine consensus IFN a (ICN), human IFN a-2a (Roferon A-Roche) and human IFN b-1a (Rebif-Serono). Experiments were run in triplicate.

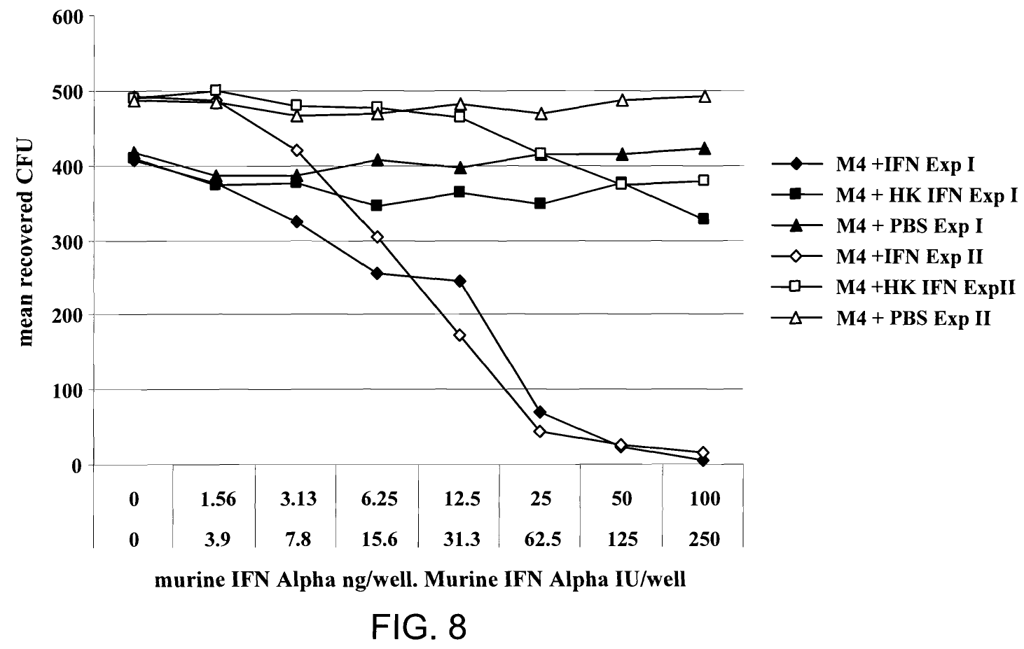

FIG. 8 shows the result from the in vitro bactericidal assay incubation of the short chain LPS Neisseria meningitides type B 44/76 Mutant 4 (M4) with murine IFN a consensus (ICN), either untreated or heat treated, using incubation at 60° C. overnight. A PBS control is included. Results of duplicate experiments are presented.

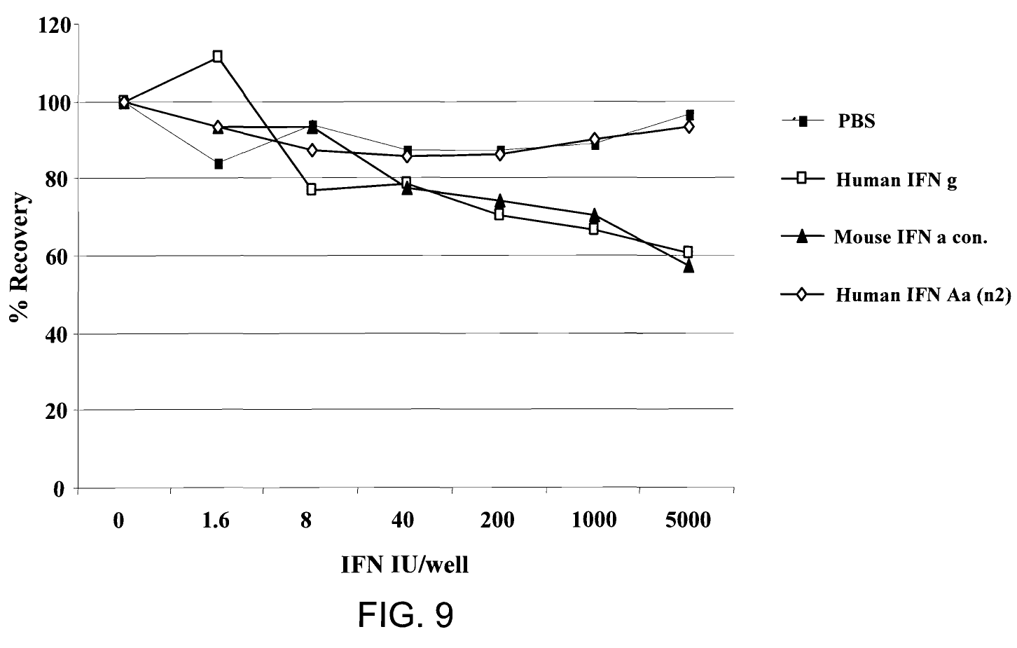

FIG. 9 shows the result from the in vitro bactericidal assay incubation of Pseudomonas aeroginosa with murine IFN A consensus (ICN), human IFN Aa (Sigma) and human IFN gamma (Sigma).

### EXAMPLE 1

### Direct Binding of Cytokines to Bacterial LPS

### Materials and Methods

Binding of cytokines (interferons and interleukins) to preparations of native (i.e. from clinical isolates) and mutant forms of bacterial LPS, either in pure form or as native outer membrane vesicles (NOMVs), was assessed by surface plasmon resonance using BIAcore™ apparatus.
The BIAcore™ systems are commercially available and accurately measure binding parameters in real-time (see www.biacore.com).

Purified, PBS-dialysed Mouse IFN alpha was obtained from ICN Biomedicals, Aurora, Ohio, USA. (Cat No. 5819C), Human IFN gamma from Sigma Aldrich, Poole, UK (Cat. No I-3265), Human IFN alpha Aa (a2) from Sigma Aldrich, Poole, UK. (I 4276), Rat IFN Beta from R and D Systems, Minneapolis, USA, (Cat. No. 13400-1 and Human IL-1a from R and D Systems, Minneapolis, USA. Cat No. (200-LA/CF)

The purified PBS-dialysed blocking antibodies-Anti-tetra His mouse IqG1 mAb, BSA-free, was obtained from QIAGEN, Hildren, Germany (Cat No. 34670), Anti-Mouse IFNa mAb cl F18, was obtained from Hycult Biotechnology, Holland and Anti-Human IFN gamma mAb, Clone 25718.11, was obtained from Sigma Aldrich, Poole, UK. Cat. No. I5521. The anti-inner core LPS mAbs used in this study were purified using protein G columns from in house hybridomas. (Andersen et al, 2002 Infection and Immunity 70:2528-2537, and Frith et al, paper in preparation).

Neisseria LPS and NOMVs are described in “Andersen S R, Guthrie T, Guile G R, Kolberg J, Hou S, Hyland L, Wong S Y Cross-reactive polyclonal antibodies to the inner core of lipopolysaccharide from Neisseria meningitidis Infect Immun. 2002 March;70(3):1293-300”.

Burkholderia LPS is described in “Jones S M, Ellis J F, Russell P, Griffin K F, Oyston P C. Passive protection against Burkholderia pseudomallei infection in mice by monoclonal antibodies against capsular polysaccharide, lipopolysaccharide or proteins. J Med Microbiol. 2002 December;51(12):1055-62”. Tularaemia LPS is described in “Prior J L, Prior R G, Hitchen P G, Diaper H, Griffin K F, Morris H R, Dell A, Titball R W. Characterization of the O antigen gene cluster and structural analysis of the O antigen of Francisella tularensis subsp. tularensis. J Med Microbiol. 2003 October;52(Pt 10):845-51”.

Yersinia (Plague) LPS is described in “Prior J L, Hitchen P G, Williamson D E, Reason A J, Morris H R, Dell A, Wren B W, Titball R W. Characterization of the lipopolysaccharide of Yersinia pestis. Microb Pathog. 2001 February;30(2):49-57”.

For comparative purposes antibodies know to bind specifically to the LPS/NOMVs under test were evaluated in parallel with the cytokines. Palmitoyl oleoyl phosphatidylcholine (POPC) was used as a blank surface control for non-specific binding.

Surface plasmon resonance (SPR) allows the direct measurement of analyte binding to a surface-bound ligand in real time. A significant advantage of this is that it provides data regarding both on (ka) and off (kd) rates, in addition to the equilibrium constants (KA/D). The kinetics of cytokine/antibody interaction with lipopolysaccharide (LPS) from a variety bacterial strains were determined using lipophilic surfaces (L1 chips #BR-1005-43) in a Biacore 3000 instrument (Biacore, Uppsala, Sweden).

Liposomes adhere spontaneously to these surfaces and can form stable bilayers, thus mimicking the in vivo environment and presentation of ligand to analyte.

All samples were diluted in standard HBS-N buffer [0.01 M HEPES pH 7.4, 0.15 M NaCl buffer (#BR-1003-69)], which was also used as the running buffer. All buffers were degassed and filter sterilised through 0.2 μm filters prior to use.

A variety of LPS-containing surfaces were created and examined. POPC (2-Oleoyl-1-palmitoyl-sn-glycero-3-phosphocholine) (1 mg/mL stock diluted 1:100 in HBS-N) was used throughout as a control surface for non-specific interactions. In addition to purified LPS (1 mg/mL stock diluted 1:100 in HBS-N), natural outer membrane vesicles (NOMVs) (1 mg/mL stock diluted 1:25 in HBS-N to give approximately equivalent LPS concentrations), and psuedo-NOMV's (LPS mixed at 1:4 mole ratio with POPC) were also immobilised. For the LPS and POPC surfaces,

unilamellar liposomes with a relatively defined size were produced by extrusion of multilamellar liposomes through a 50 nm membrane, prior to passing the solution over the lipophilic L1 surface at 5 μL/min. Approximately 1500 response units (RU) was immobilised for each surface.

For the kinetic studies, the machine temperature was maintained at 21° C., and high flow rates (30 μl/min) were used throughout, to minimise surface mass transfer effects. Each analyte was passed over the sensor surface for 3 minutes using the standard kinetics inject (KINJECT) method, with dissociation data collected for a further 10 minutes prior to regeneration. The surface was regenerated between each interaction analysis, using 20 mM CHAPS to completely remove all bound liposomes from the sensor surfaces, and then the desired surface was recreated as described above.

### Results

The BIAcore™ apparatus was used to analyse the real time liquid binding of the IFNs and other cytokines to bound LPS and NOMVs. It was shown that the kinetic binding on rate, elution off rate and overall KD for LPS and NOMV of the murine IFN alpha is similar to a number of mAbs that were raised against inner core LPS epitopes on an NOMV vaccine (see table 1 and 2 below).

TABLE 1
BIAcore binding results for purified antibodies and interferons
binding to N. meningitidis Mutant 4 LPS and NOMV'S.

| | Mutant | Mutant | Mutant | Mutant 4 | |
|---|---|---|---|---|---|
| | 4 LPS | 4 LPS | 4 LPS | LPS | Mutant 4 |
| | ka | kd | ka/kd = KA | 1/Ka = KD | NOMV |
| | (1/Ms) | (1/s) | (1/M) | (M) | KD (M) |
| Mouse Antibody | | | | | |
| (100 nM) | | | | | |
| 216H11 | 1.82E+5 | 1.43E−3 | 1.27E+8 | 7.85E−9 | 4.62E−9 |
| Fc12 | 9.23E+4 | 1.66E−4 | 5.56E+8 | 1.80E−9 | 1.82E−9 |
| HG7 Pool | 1.31E+5 | 1.88E−5 | 6.96E+9 | 1.44E−10 | 6.41E−10 |
| HG7#2 | 1.33E+5 | 9.33E−5 | 1.43E+9 | 7.01E−10 | 1.61E−9 |
| HG7#3 | 1.15E+5 | 7.41E−5 | 1.55E+9 | 6.47E−10 | 1.45E−9 |
| Sheep Antibody | | | | | |
| (100 nM) | | | | | |
| DC8 | 1.29E+5 | 2.56E−4 | 5.04E+8 | 1.98E−9 | ND |
| DG8 | 1.22E+5 | 3.72E−4 | 3.28E+8 | 3.05E−9 | ND |
| EF5 | 2.25E+5 | 2.86E−3 | 7.87E+7 | 1.27E−8 | ND |
| Interferons | | | | | |
| (100 nM) | | | | | |
| Bovine IFN | 6.19E+3 | 6.27E−3 | 9.86E+5 | 1.01E−6 | 1.62E−5 |
| Alpha | | | | | |
| Mouse IFN | 1.37E+5 | 2.82E−4 | 4.86E+8 | 2.06E−9 | 8.84E−8 |
| Alpha con | | | | | |
| Rat IFN Beta | 8 | 2.54E−4 | 3.03E+4 | 3.30E−5 | 3.85E−6 |

TABLE 2
BIAcore binding results for Human IFN Gamma and Human IFN Alpha a (a2) binding
to LPS from the short chain LPS mutant 4 (M4) and the clinical isolates
240101 (101) and 240013 (013) of Neisseria meningitides (type B).

| | ka (1/Ms) | kd (1/s) | KA (1/M) | KD (M) | Chi2 | Rmax (RU) |
|---|---|---|---|---|---|---|
| Human IFN gamma | | | | | | |
| [100 nM] | | | | | | |
| M4 LPS | *** | *** | *** | *** | *** | *** |
| 101 LPS | 4.04E+04 | 1.09E−04 | 3.71E+08 | 2.70E−09 | 8.93E+00 | 1.11E+03 |
| 013 LPS | 2.41E+04 | 1.36E−03 | 1.77E+07 | 5.66E−08 | 0.188 | 25.3 |
| Human IFN | | | | | | |
| alpha a | | | | | | |
| M4 LPS | 1.19E+05 | 3.89E−04 | 3.06E+08 | 3.27E−09 | 3.56E−01 | 84.5 |
| 101 LPS | *** | *** | *** | *** | *** | *** |
| 013 LPS | 2.04E+05 | 1.13E−03 | 1.80E+08 | 5.55E−09 | 0.515 | 50.3 |

xxx no binding

TABLE 3
BIAcore results for Interferons and Interleukins
binding to Franscicella tularensis LPS

| | | ka | kd | ka/kd = KA | 1/Ka = KD |
|---|---|---|---|---|---|
| | nM analyte | (1/Ms) | (1/s) | (1/M) | (M) |
| Interferons | | | | | |
| Human IFN A | NIBSC | 1.45E+6 | 3.33E−4 | 4.28E+9 | >3.55E−10 |
| con. | 5, 2.5, | | | | |
| | 1.25 nM | | | | |
| Human IFN B. | NIBSC 10, | 3.65E+6 | 1.23E−3 | 2.96E+9 | 3.76E−10 |
| | 5, 2.5 nM | | | | |
| Human IFN A | NIBSC 10, | 1.93E+6 | 8.57E−4 | 2.44E+9 | 6.22E−10 |
| leuc. | 5, 2.5 nM | | | | |
| Interleukins | | | | | |
| Interleukin- | NIBSC 5, | 1.86E+6 | 3.86E−4 | 4.52E+9 | >3.07E−10 |
| 4 | 2.5, 1.25 nM | | | | |
| Interleukin- | NIBSC 10, | 1.97E+6 | 5.46E−4 | 3.44E+9 | 3.88E−10 |
| 5 | 5, 2.5 nM | | | | |
| Interleukin- | NIBSC 10, | 2.53E+6 | 7.87E−4 | 3.13E+9 | 3.53E−10 |
| 6 | 5, 2.5 nM | | | | |

TABLE 4
BIAcore binding results for Interferons and Interleukins
binding to Burkholderia pseudomallei LPS.

| | | | | ka/kd = KA | 1/Ka = KD |
|---|---|---|---|---|---|
| | nM analyte | ka (1/Ms) | kd (1/s) | (1/M) | (M) |
| Interferons | | | | | |
| Human IFN A | NIBSC | 1.13E+6 | 2.34E−4 | 4.52E+9 | >2.65E−10 |
| con. | 5, 2.5, | | | | |
| | 1.25 nM | | | | |
| Human IFN B. | NIBSC 10, | 9.86E+6 | 3.41E−3 | 7.51E+8 | 9.67E−8 |
| | 5, 2.5 nM | (1.44E+5) | | (4.17E+7) | (40×) |
| | | | | | 2.39E−8 |
| | | | | | (160×) |
| Human IFN A | NIBSC 10, | 1.16E+6 | 6.74E−4 | 1.76E+9 | 9.05E−10 |
| leuc. | 5, 2.5 nM | | | | |
| Interleukins | | | | | |
| Interleukin-4 | NIBSC 5, | 1.40E+6 | 2.62E−4 | 4.93E+9 | >2.45E−10 |
| | 2.5, 1.25 nM | | | | |
| Interleukin-5 | NIBSC 10, | 2.11E+6 | 5.46E−4 | 3.53E+9 | 3.69E−10 |
| | 5, 2.5 nM | | | | |
| Interleukin-6 | NIBSC 10, | 3.51E+6 | 8.78E−4 | 3.89E+9 | 3.50E−10 |
| | 5, 2.5 nM | | | | |

The results obtained for Burkholderia pseudomallei (table 4) indicated that NIBSC Interferon Alpha Consensus, NIBSC Interferon beta (B), NIBSC Interferon Alpha leuco, NIBSC Interleukin 4 and NIBSC Interleukin 5 bind to the purified LPS.

The Tularaemia LPS binding data (Table 3) is significantly different: Under identical assay conditions NIBSC Human Interferon Beta is able to bind this LPS with high affinity. This indicates a degree of specificity of certain Interferons for certain LPS forms.

### EXAMPLE 2

### In Vitro Killing Assay

The ability of various cytokines to directly kill bacteria in the absence of any other immune system components was assessed in an in vitro killing assay.

### Materials and Methods

An in vitro killing assay has been set up using lab strains, genetic mutants and clinical isolates of N. meningitidis type B. The assay is a modification of that described in S. typhimurium by Howells A. M., et al., Res Microbiol. 2002, June 153(5), pp 281-7.

Briefly, bacterial colonies from overnight streaked plates were re-suspended in 100 ml of an appropriate medium for the bacteria under test (BHI for N. meningitides) and grown for 3 hours at 37° C., 140 rpm. Log phase cultures were then pelleted, re-suspended, washed in media (×2) and PBS (×3) and OD adjusted to 546 nm=0.600+/−0.01 (equivalent to 1-5×10⁷ CFU/ml). A series of 1/10 dilutions were prepared from this stock culture. A series of 1/10 etc. dilutions were also prepared for each reagent under test (e.g. cytokines and antibody controls). Aliquots of 20 μl diluted bacteria and 50 μl diluted reagent were transferred to each well of a 96 well assay plate together with 180 μl PBS per well.

Plates were then incubated for 3 hours at 37° C., 5% CO₂. Four 50 μl aliquots from each well were diluted and plated out on solid media appropriate for the bacterium under test (BHI+1% HS plates from N. meningitides type B) and incubated overnight at 37° C., 5% CO₂. Colonies were counted as an indication of the numbers of viable bacteria, relative to colonies recovered in PBS-treated wells and untreated wells (for viable count purposes).

### Results

Reproducible dose-responsive killing was observed using recombinant purified (95%+purity) human, mouse and rat IFNs (both from academic and commercial sources and expressed in either bacteria or mammalian cell cultures). It is possible to negate the killing effects of murine IFN alpha and human IFN gamma by using specific monoclonal antibodies that bind IFNs directly or bind inner core LPS.

The following are representative results:

### (1) Human Interferon Gamma

Tested against N. meningitidis type B clinical isolate 240 101.

### The Antibodies Used Were

(i) a commercially available neutralizing anti-Human IFN-Gamma mAb (nmab)—labelled anti-IFN γ in FIG. 1.
(ii) HH9, an anti-NmB inner core LPS binding mAb
(iii) Anti-tetra His mAb—an isotype control mAb that does not bind either LPS or IFN gamma—labelled anti-Tetra in FIG. 1.

All blocking antibodies were used as a 1:10 dilution of 1 mg/ml stocks, 20 μl/well.

Results are presented in FIG. 1 as the percentage recovery of bacteria plotted against levels of IFN gamma, which additionally includes a PBS control.

(2) Human IFN Alpha aA (n2)
Tested against N. meningitidis type B clinical isolate 240 101 and the short chain LPS mutant 4.

Results are shown in FIG. 2 as the percentage recovery of bacteria plotted against levels of IFN (both units and weight per well).

### (3) Purified Recombinant Murine Consensus Interferon Alpha

Tested against N. meningitidis type B clinical isolate 240 101.

### The Antibodies Used Were:

(i) a commercially available neutralizing murine IFN-alpha mAb F18(nmAb)—labelled in FIG. 3a as F18 and in FIG. 3b as nmAb,
(ii) HH9, an inner core LPS-binding mAb—labelled as HH9 in FIG. 3b,
(iii) Anti-tetra His mAb—an isotype control mAb that does not bind either LPS or IFN—labelled Tetra in FIG. 3b. Results are shown in FIG. 3a (F18 only) and 3b (in the presence of either F18mAb or competing anti-LPS mAbs used at 1:10 and 1:100 dilutions). Again, a PBS control lane is included and percentage recovery of bacteria is plotted against levels of consensus murine IFN alpha (weights and units per well).

### (4) Purified Recombinant Rat IFN Beta

Tested against N. meningitidis type B clinical isolate 240 101.

Results are shown in FIG. 4b.

Results of IFN Beta binding to various LPS molecules using BIAcore apparatus is shown in FIG. 4a. Here;
Fc1=POPC (control)
Fc2=LPS from N. meningitidis type B Mutant 4
Fc3=LPS from N. meningitidis type B clinical isolate 240 101.
Fc4=LPS from N. meningitidis type B clinical isolate 240 013

### (5) Purified Recombinant Human IL-1a

Tested against N. meningitidis type B LPS short chain mutant 4.

### The Antibodies Used Were:

(i) HH9, an inner core LPS-binding mAb—labelled HH9 in FIG. 5.
(ii) Anti-tetra His mAb—an isotype control mAb that does not bind either LPS or IFN—labelled Anti-His in FIG. 5.

Results are presented in FIG. 5, with percentage bacterial recovery plotted against levels of Human interleukin la (IL-1a). A PBS control is included.

(6) Murine Consensus IFN a (ICN), Human IFN a-2a (Roferon A from Roche) and Human IFN b-1a (Rebif from Serono).
Tested against the short chain LPS Neisseria meningitidis type B 44/76 mutant 4 (M4). A PBS control is included.

Results from the in vitro bactericidal assay are presented in FIG. 6.

(7) The Murine Consensus IFN a (ICN), Human IFN a-2a (Roferon A -Roche) and Human IFN b-1a (Rebif-Serono).
Tested against the Neisseria meningitidis type B clinical isolate 240 101.

Experiments were run in triplicate. Results from the in vitro bactericidal assay are shown in FIG. 7.

### EXAMPLE 3

### Serum Bactericidal Assay (SBA)

Briefly, a bacterial innoculum was incubated in the presence of 25% human complement in microtitre plates, two-fold dilution series of specific purified IFN's and mabs were tested for serum bactericidal activity (SBA) against the NmB short chain LPS mutant 4 (Andersen S R et al, 1995. Microb. Pathog.19: 159-168) and incubated for 0, 1, 2 or 3 hours at 37° C., 5% CO₂. Aliquots from each well were diluted and plated out on solid media appropriate for the bacterium under test (BHI+1% HS plates from N. meningitides type B) and incubated overnight at 37° C., 5% CO₂. Colonies were counted and the results are presented as the SBA titre which is defined as the reciprocal of the highest dilution in serum causing more than 50% killing of the target strain. The SBA method used is described in Findlow J et al, 2005. Vaccine 28; 2623-2627 and was performed by Jamie Findlow, Vaccine Evaluation Department, HPA North West, Manchester Laboratory, Manchester Medical Microbiology Partnership, PO Box 209, Clinical Sciences Building II, Manchester Royal Infirmary, Manchester. M13 9WZ, UK.

TABLE 6
Results

| | t0^{(a)} | t60 | t120 | t180 | t240 |
|---|---|---|---|---|---|
| mAb^{(b)} | | | | | |
| DC8 | 2^{(d)} | 2 | 2 | 2 | 2 |
| DG8 | 2 | 2 | 2 | 2 | 2 |
| EF5 | 2 | 2 | 2 | 2 | 2 |
| EH9 | 2 | 2 | 2 | 2 | 2 |
| Interferon | | | | | |
| Mo IFNa | 2 | 2 | 4 | 4^{(e)} | 4 |
| ICN^{(c)} | | | | (625 ng)^{(f)} | |
| Hu IFNaA | 4 | 4 | 8 | 8 (3 ng) | 4 |
| (a2) | | | | | |
| Rat IFNb | 2 | 2 | 8 (20.5 ng) | 4 (41.0 ng) | 4 |

Key to superscripted letters:
^{(a)}Modified serum bactericidal assay (SBA) incubation time in minutes.
^{(b)}Sheep monoclonal antibodies (mAbs) (1 mg/ml) previously negative in 3 independent SBA's.
^{(c)}Mouse mixed subtype IFN a.
^{(d)}Reciprocal dilution.
^{(e)}In bold = complement-independent killing.
^{(f)}approximate concentrations of Interferon.

The SBA assay provides an independent validation of the in vitro killing assay and demonstrated complement-independent killing of the bacteria after a prolonged incubation period in the presence of mouse IFN Alpha consensus, human IFN Alpha a2 and Rat IFN Beta.

### EXAMPLE 4

### The Direct Bactericidal Activity of Cytokines is Dependent Upon the Presence of Bioactive Protein

In this experiment, the bactericidal assay described in Example 2 was carried out using the short chain LPS Neisseria meningitides type B 44/76 Mutant 4 (M4). Against this strain were tested murine IFN a consensus (ICN), either untreated or heat treated, using incubation at 60° C. overnight.

The results in FIG. 8 clearly show that bioactive protein is required in order to mediate direct bacterial killing. A PBS control is included. Results of duplicate experiments are presented.

### EXAMPLE 5

### Specific Cytokines are Capable of Mediating Direct Killing of Pseudomonas Aeroginosa

In this experiment, the bactericidal assay described in Example 2 was carried out using murine IFN A consensus (ICN), human IFN Aa (Sigma) and human IFN gamma (Sigma) against the Pseudomonas aeroginosa G38 strain isolated from a patient with sepsis.

As is shown by the results presented in FIG. 9, both murine IFN A consensus (ICN) and human IFN gamma (Sigma) were able to mediate direct killing of Pseudomonas aeroginosa. Human IFN Aa (n2) on the other hand was not capable of causing direct killing.

1. A method of screening a cytokine or a derivative or mimetic thereof for the ability to kill directly a bacterium comprising determining whether the cytokine or derivative or mimetic thereof has the ability to bind to LPS from the bacterium.

2. The method of claim 1 wherein the LPS is found as a component of Native Outer Membrane Vesicles (NOMVs) or whole bacterial cells.

3. The method of claim 1 wherein surface plasmon resonance is utilised in order to determine whether the cytokine or derivative or mimetic thereof binds to LPS.

4. A method of screening a cytokine or a derivative or mimetic thereof for the ability to kill directly a bacterium comprising;
a) culturing suitable bacterial cells in the presence of the cytokine or derivative or mimetic thereof and in the absence of any components of the immune system; and
b) determining the number of viable bacterial cells.

5. The method of claim 1, further comprising:
a) culturing suitable bacterial cells in the presence of the cytokine or derivative or mimetic thereof and in the absence of any components of the immune system; and
b) determining the number of viable bacterial cells.

6. The method of claim 4 wherein the components of the immune system include B cells, T cells, phagocytes, serum and/or complement.

7. A vaccine composition comprising pathogenic bacterial cells and/or native outer membrane vesicles and at least one cytokine, wherein the cytokine is capable of binding to a lipopolysaccharide present on the bacterial cells and/or native outer membrane vesicles and/or mediating direct killing of the bacteria.

8. A vaccine composition according to claim 7 which comprises native outer membrane vesicles from Neisseria meningitides, preferably from Neisseria meningitidis type B and at least one cytokine which is capable of binding to a lipopolysaccharide on said native outer membrane vesicles.

9. A vaccine composition according to claim 7 which comprises native outer membrane vesicles from Pseudomonas aeroginosa and at least one cytokine which is capable of binding to a lipopolysaccharide on said native outer membrane vesicles.

10. A vaccine composition according to claim 8 wherein the cytokine is an interferon.

11. A method of preparing a vaccine comprising contacting a preparation comprising live pathogenic bacterial cells and/or native outer membrane vesicles with at least one cytokine, wherein the cytokine is capable of binding to a lipopolysaccharide of the bacteria and/or native outer membrane vesicles and/or mediating direct killing of any bacteria present in the preparation.

12. A method according to claim 11 wherein the preparation comprises native outer membrane vesicles from Neisseria meningitides, preferably Neisseria meningitidis type B.

13. A method according to claim 11 wherein the preparation comprises native outer membrane vesicles from Pseudomonas aeroginosa.

14. A method according to claim 13 wherein the cytokine is an interferon.

15. A method of screening human serum for innate immunity to bacterial infection, the method comprising assaying the serum for the presence of one or more cytokines capable of binding to a lipopolysaccharide present on a bacterial cell or a bacterial cell membrane component and/or mediating direct killing of the bacteria, wherein the presence of one or more of said cytokines is taken as an indication of innate immunity to bacterial infection.

16. A method of screening human serum for the ability to kill bacterial cells, the method comprising incubating the serum with bacterial cells in the absence of any immune cells or complement then assaying the number of viable bacterial cells present.

17.-30. (canceled)

31. A method of treating or preventing bacterial infection in a mammalian host, the method comprising administering to said host an effective amount of at least one cytokine, wherein the cytokine mediates direct killing of the bacteria.

32. A method of neutralising or directing for removal circulating lipopolysaccharide or cell membrane-derived vesicles or components comprising lipopolysaccharide in a mammalian subject, the method comprising administering to said subject an effective amount of at least one cytokine.

33. A method according to claim 32 wherein at least one cytokine is an interferon or an interleukin.

34. A method according to claim 33 wherein the interferon is IFN alpha, IFN beta or IFN gamma.

35. A method according to claim 34 wherein the interferon is a mammalian interferon.

36. A method according to claim 35 wherein the interferon is murine, rat or human interferon.
